(19) United States
(12) Patent Application Publication
(10) Pub. No.: US 2020/0315337 A1
(43) Pub. Date: Oct. 8, 2020

(54) USE OF AN APPLICATOR IN FREE ROTATION FOR THE MAKEUP REMOVAL OF MASCARA

(71) Applicant: LO'REAL, Paris (FR)

(72) Inventors: Philippe ILEKTI, Chevilly La Rue (FR); Alexis LEONARD, Clichy (FR); Marion FRARCHE, Chevilly La Rue (FR)

(21) Appl. No.: 16/955,489

(22) PCT Filed: Dec. 20, 2018
(86) PCT No.: PCT/EP2018/086425

§ 371 Date: Jun. 18, 2020

(30) Foreign Application Priority Data
Dec. 20, 2017 (FR) 1762605

(51) Int. Cl. A46B 13/08; A45D 34/04; A46B 9/02; A61K 8/73; A61K 8/34; A61K 8/44; A61K 8/46; A61Q 1/14; A61Q 1/10

(52) U.S. Cl. CPC A46B 13/08; A45D 34/043; A45D 34/045; A46B 9/021; A46B 9/025; A61K 8/731; A46B 2200/1046; A61K 8/442; A61K 8/44; A61K 8/463; A61Q 1/14; A61Q 1/10; A45D 2200/1063; A61K 8/345

(57) ABSTRACT

Use of an applicator in free rotation for the makeup removal of mascara The invention also has for object a cosmetic application requisite comprising: —an application member able to be driven in rotation by the keratin fibers with respect to a gripping member; and —makeup removal composition for keratin fibers comprising an aqueous phase and at least one surfactant. It also has for object a method for applying this requisite.

This invention relates to a makeup removal requisite, as well as a method using it.

Removing makeup from the area of the eye is very delicate. It must be as effective as possible, while rubbing the least possible as this zone is very sensitive. Indeed, the fatty residues such as excess sebum or the remainder of cosmetic products used daily, can accumulate in the skin folds and on the surface of the skin, leaving an unsightly aspect on the skin.

Makeup removers exist today packaged in the form of a squeezy bottle, that is applied with an applicator for eyelashes. This type of application makes it possible to deposit the makeup remover locally; the makeup removal obtained is thus cleaner and less aggressive than a makeup removal carried out directly with cotton pads. However, this type of makeup removal is not very comfortable: indeed, the passing of an applicator on the eyelashes covered with a dry deposit of mascara is not very pleasant due to the rigidity of the deposit and requires several passes in order to deposit a sufficient quantity of makeup remover on the eyelashes.

There is therefore a need for a method for the makeup removal from keratin fibers, in particular from eyelashes, that can be used locally on said fibers, in particular said eyelashes, and that is comfortable and easy to implement.

This invention makes it possible to resolve this problem: it indeed facilitates the application of a makeup remover, and this, directly and locally on the coated eyelashes.

The invention also has for object an application requisite comprising:
an application member able to be driven in rotation by the keratin fibers with respect to a gripping member; and
makeup removal composition for keratin fibers comprising an aqueous phase and at least one surfactant.

Indeed, as shown as examples, the use of such an application member makes it possible to deposit the makeup remover with reduced forces of friction. The deposit of makeup remover on the eyelashes is therefore more comfortable and easier for the user.

The application requisite according to the invention can also comprise at least one coating composition of keratin fibers.

The requisite according to the invention can comprise one or more of the following features, taken alone or in any technically possible combination:
the driving in rotation of the application member with respect to the gripping member is carried out around a longitudinal axis;
the application member comprises at least one first row of pins and at least one second row of pins, at least one picot of the second row being arranged axially along the longitudinal axis between two adjacent pins of the first row;
the application member comprises a third row of pins, each pin of the first row being axially positioned, along the longitudinal axis, at the same position as a pin of the third row;
in projection in a plane perpendicular to the longitudinal axis, each pin of the second row of pins is arranged between pins of the first row and pins on the third row, preferably in the middle of the pins of the first row and pins of the third row;
the application member comprises a plurality of rows of pins, each row having at least one axially positioned pin, along the longitudinal axis, at the same position as a pin of each one of the other rows of pins;
each pin of at least one row of pins is axially positioned, along the longitudinal axis, at the same position as a pin of each one of the other rows of pins;
the rows of pins extend parallel to the longitudinal axis;
the application member comprises a foam;
the application member is made of elastomer;
each pin of the second row is located midway axially along the longitudinal axis of two adjacent pins of the first row;
the difference between two adjacent pins of the same row is identical for all of the pins of the row and for all of the rows;
the pins have the same height, the difference between two adjacent pins of the same row being between 1% and 10% of the height of the pins, said difference being taken on an outer surface of a support body on which the rows of pins are added;
the pins have the same height, the difference between a pin of the second row and at least one pin of the first row being less than 10% of the height of the pins, said difference being taken on an outer surface of a support body on which the rows of pins are added;
the makeup removal phase comprises at least 30% by weight of water with respect to the total weight of the composition;
surfactant of the makeup removal composition is chosen from:

a) anionic surfactants;

b) amphoteric surfactants;

c) non-ionic surfactants, and

d) mixtures thereof;
the cosmetic application requisite also comprises a coating composition of keratin fibers.

The invention also has for object a process for applying a makeup removal composition on keratin fibers comprising the following steps:
supply of a cosmetic application requisite according to the invention;
impregnation of the application member with the makeup removal composition;
bringing the application member in contact with keratin fibers;
driving in rotation of the application member with respect to the gripping member by displacement of the application member on the keratin fibers; and,
depositing of the makeup removal composition on the keratin fibers.

The method according to the invention can comprise one or more of the following features, taken alone or in any technically possible combination:
the keratin fibers are coated with a coating composition, at least before the bringing the application member in contact with keratin fibers;
the coating composition comprises at least 30% by weight of water with respect to the total weight of the composition.

Applicator

The description of a requisite for application able to implement the process for applying according to the invention given solely as an example, is done in reference to the accompanying drawings, wherein:

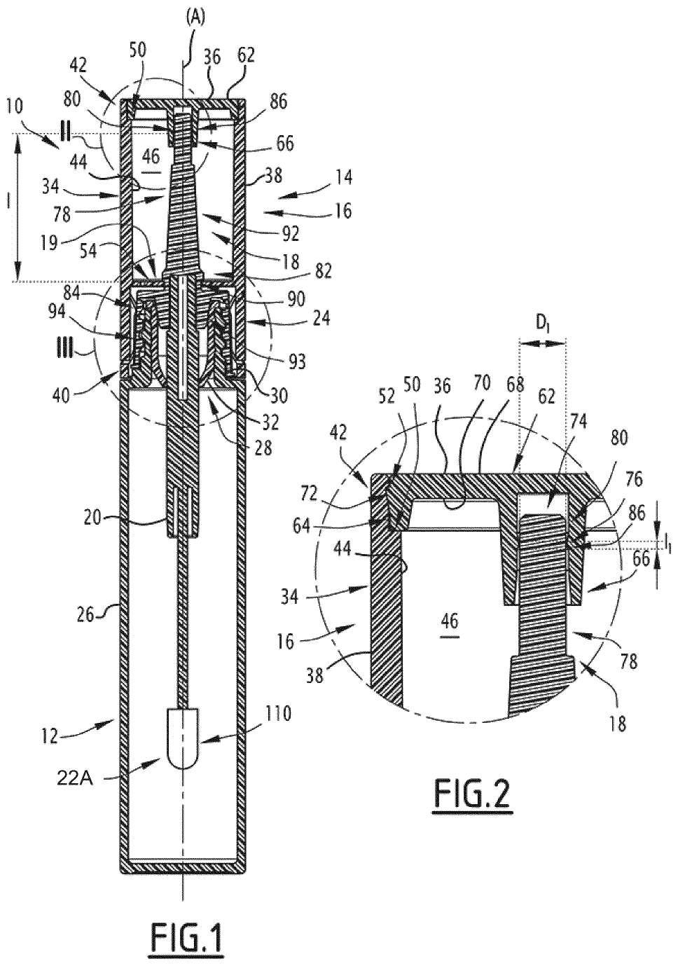

FIG. 1 is a diagrammatical view as a longitudinal cross-section of a device for a packaging and application device for a makeup removal composition of the base composition of a requisite according to the invention, with the applicator being mounted onto the receptacle;

FIG. 2 is a diagrammatical view as a longitudinal cross-section showing a first linear contact region between the rod carrier and the gripping member of the device in FIG. 1;

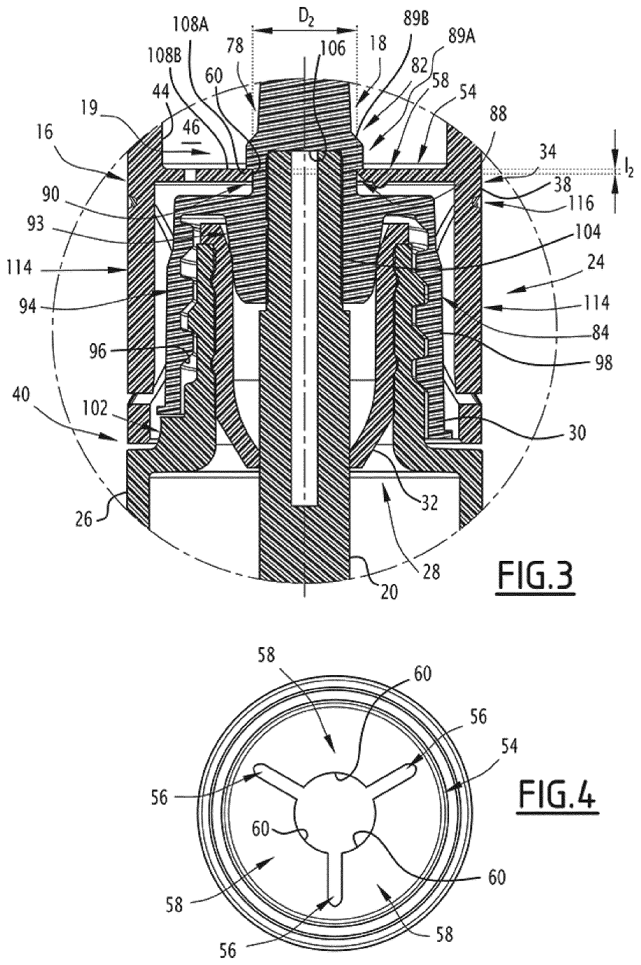

FIG. 3 is a diagrammatical view as a longitudinal cross-section of a second linear contact region between the rod carrier and the gripping member of the device in FIG. 1;

FIG. 4 is a diagrammatical view as a transverse cross-section of contact tongues defining the second contact region of the device in FIG. 1;

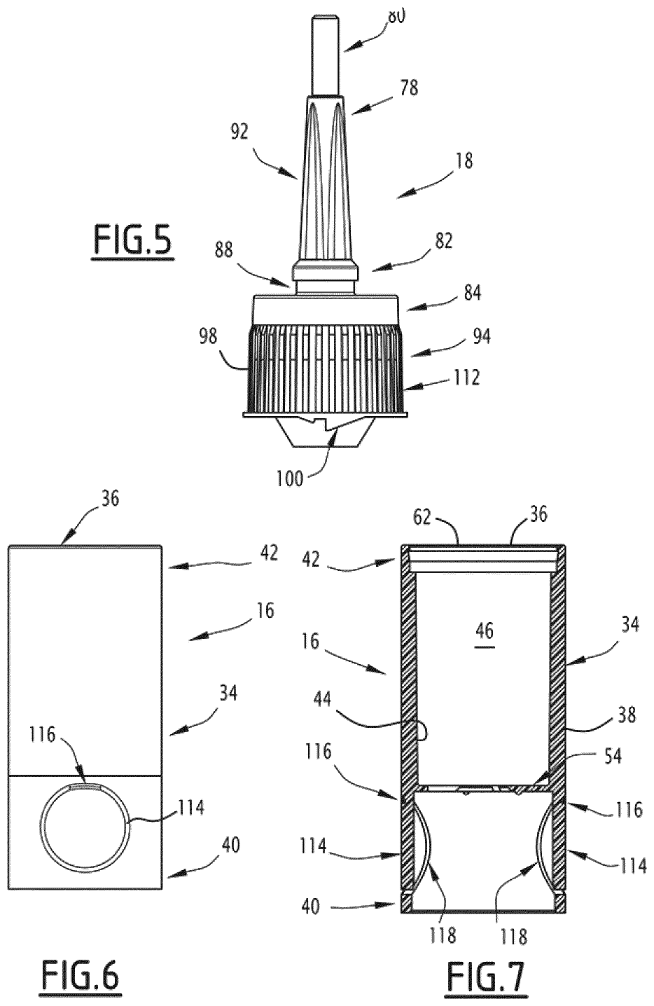

FIG. 5 is a diagrammatic view in elevation of the rod carrier of the device in FIG. 1;

FIG. 6 is an outside diagrammatical view of the gripping member of the device of FIG. 1;

FIG. 7 is a sectional diagrammatical view along a median plane of the gripping member of the device in FIG. 1;

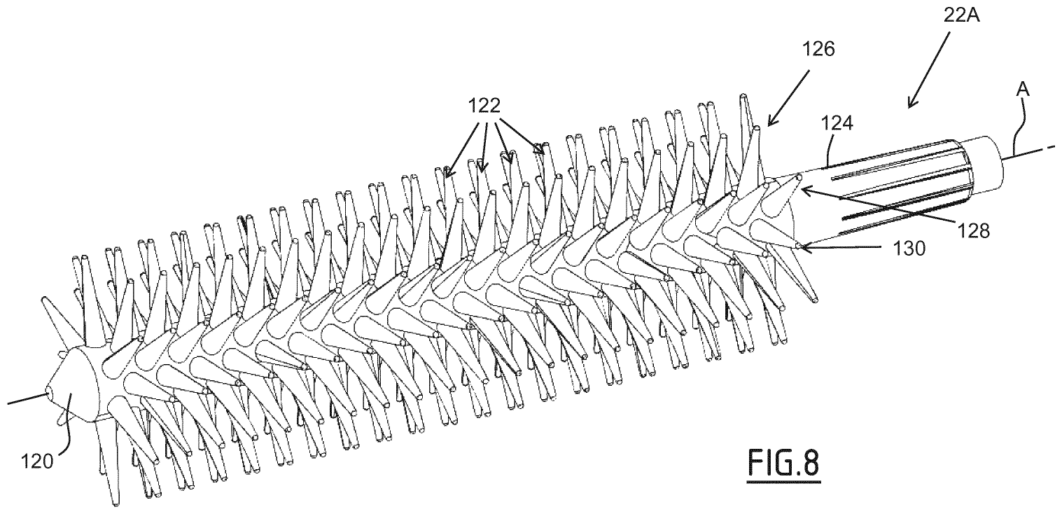

FIG. 8 is a diagrammatical view in perspective of a first application member of the packaging device of FIG. 1;

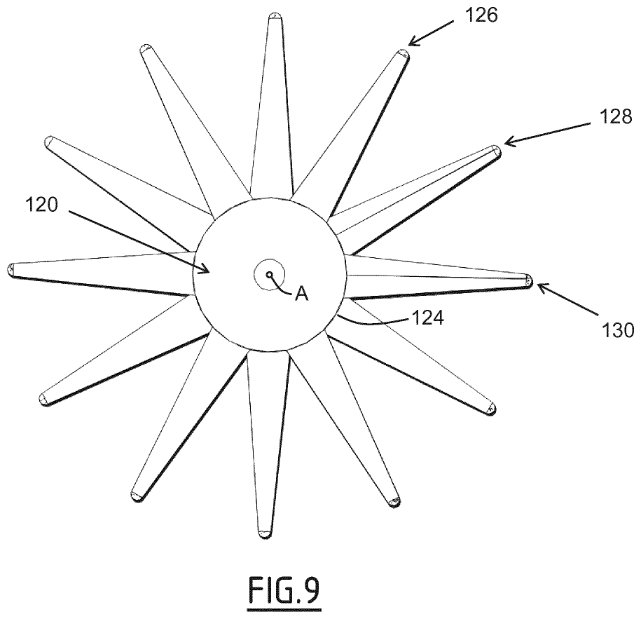

FIG. 9 is an axial diagrammatical view of the application member of the device of FIG. 8; and

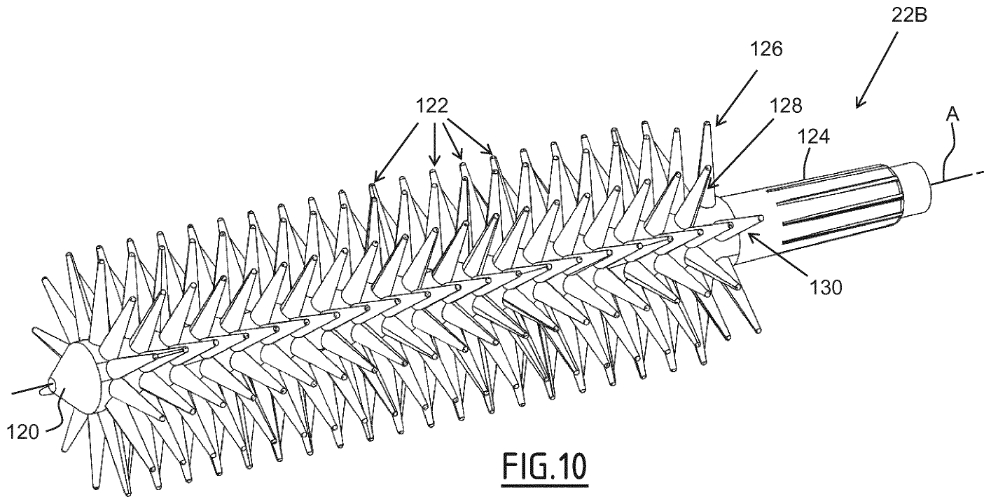

FIG. 10 is a diagrammatical view in perspective of a second application member of the packaging device of FIG. 1.

An application requisite according to the invention comprises a device 10 for packaging and applying a makeup removal composition on keratin fibers and a makeup removal composition for keratin fibers.

Preferably, the application requisite also comprises a coating composition of keratin fibers.

The keratin fibers are typically eyelashes. The makeup removal composition is then a makeup removal composition of eyelashes. This makeup removal composition of eyelashes shall be described following the description of the requisite for application.

A first embodiment of the device 10 for packaging and applying a makeup removal composition is shown in FIGS. 1 to 9.

The device 10 comprises a receptacle 12 intended to contain the makeup removal composition of the base composition, and an applicator 14 removably mounted on the receptacle 12 and able to apply the makeup removal composition.

The device 10 advantageously has a generally cylindrical shape extending along a longitudinal axis A. The terms “longitudinal” and “radial” generally mean in relation to the longitudinal axis A.

In reference to FIG. 1, the receptacle 12 comprises a hollow body 26, able to contain the makeup removal composition, and a threaded neck 30 protruding outwards from the body 26 by defining an access opening 28 for the product.

The receptacle 12 also comprises a squeezing device 32 inserted into the neck 30.

The applicator 14 comprises a gripping member 16, extending along the longitudinal axis A, a rod carrier 18, arranged in the gripping member 16 and mounted freely mobile in rotation about the longitudinal axis A with respect to the gripping member 16.

The applicator 14 also comprises a blocking device 19 that opposes the translation along the axis A of the rod carrier 18 with respect to the gripping member 16, a rod 20 integral with the rod carrier 18, a system for the selective blocking in rotation 24 of the rod carrier 18 with respect to the gripping member 16, and a first application member 22A of the makeup removal composition (FIGS. 8 and 9) carried by the rod 20.

The applicator 14 is mounted mobile between an idle position, wherein is mounted on the recipient 12, the rod 20 and the first application member 22A being engaged at least partially in the receptacle 12, and a usage position, in which the rod 20 and the first application member 22A are extracted outside of the receptacle 12.

In the idle position, the applicator 14 is maintained fixed on the receptacle 12 for example by screwing on the neck 30.

The gripping member 16 comprises a cover 34 and a plug 36, with the plug 36 here added on the cover 34.

The cover 34 has a shape of revolution about the longitudinal axis A. It is for example cylindrical.

The cover 34 comprises a peripheral wall 38 about the longitudinal axis A. It extends between a distal edge 40 intended to be placed facing the receptacle 12 in an idle position of the applicator 14, and a proximal edge 42 intended to be placed apart from the receptacle 12 in the idle position of the applicator 14.

The cover 34 is hollow, and defines an inner surface 44 that delimits an inner volume 46.

In the vicinity of the proximal edge 42 of the cover 34, the inner surface 44 of the cover 34 has a circumferential shoulder 50 of the bearing of the plug 36, shown in FIG. 2, and a protrusion 52 for maintaining the plug 36 in position against the shoulder 50.

In reference to FIGS. 3 and 4, the cover 34 comprises a transverse wall 54, that extends radially from the inner surface 44 of the cover 34.

The transverse wall 54 is arranged longitudinally apart from the proximal edge 42. It defines a plurality of radial slots 56.

The radial slots 56 delimit between them at least two contact tongues 58. The gripping member 16 here comprises three contact tongues 58.

Each tongue 58 radially protrudes from the peripheral wall 38, and more precisely, from the inner surface 44 of the cover 34.

Each tongue 58 defines a free inside edge 60, directed towards the longitudinal axis A. The free edge 60 has an inner chamfer that converges towards the axis A in the direction away from the rod 20 to the plug 36.

In FIG. 2, the plug 36 is securely fastened to the cover 34. It closes the cover 34 on its proximal edge 42.

The plug 36 comprises a sealing wall 62, a peripheral ring 64 for mounting in the cover 34 and an inner sleeve 66 for guiding the rod carrier 18.

The peripheral ring 64 protrudes from the periphery of a distal face 70 of the sealing wall 62.

The peripheral ring 64 comprises at least one attaching stop 72, for example four attaching stops 72 distributed angularly about the longitudinal axis A.

The peripheral ring 64 is arranged in abutment against the shoulder 50 of the cover 34. Each attaching stop 72 cooperates with the protrusion 52, in order to removably immobilize the plug 36 with respect to the cover 34 and provide an aesthetic aspect.

A proximal face 68 of the sealing wall 62 is flush with the proximal edge 42 of the cover 34.

The inner sleeve 66 protrudes longitudinally in the cover 34 from the distal face 70 of the sealing wall 62.

The inner sleeve 66 delimits an internal cavity 74 and a bulge 76 that protrudes radially towards the longitudinal axis A in the internal cavity 74.

The inner sleeve 66 is for example made of acrylonitrile butadiene styrene (ABS), polyoxymethylene (POM), polyethylene (PE), or polypropylene (PP).

The rod carrier 18 is for example made from polybutylene terephthalate (PBT), polyoxymethylene (POM) or polypropylene (PP).

The rod carrier 18 comprises a longitudinal guiding segment 78, extending between a proximal end 80 and a distal end 82, and a head 84 for mounting on the receptacle 12.

The longitudinal segment 78 extends in the internal volume 46 along the longitudinal axis A. The proximal end 80 is received in the internal cavity 74 defined by the inner sleeve 66.

The rod carrier 18 as such has, on the bulge 76, a first linear contact region 86 with the gripping member 16. The first linear contact region 86 is a circumferential linear contact region about the longitudinal axis A.

In the example shown, the bulge 76 is continuous in the circumferential direction. The first linear contact region 86 of the rod carrier 18 is as such also continuous in the circumferential direction. Alternatively, the bulge 76 and/or the rod carrier in the first linear contact region is (are) discontinuous in the circumferential direction. The first linear contact region 86 of the rod carrier 18 is as such advantageously discontinuous in the circumferential direction.

The longitudinal segment 78 has, on the first contact region 86, a maximum diameter D1. The maximum diameter D1 is, advantageously between 1.5 mm and 5 mm, in particular between 2.0 mm and 3 mm.

The longitudinal segment 78 also defines a circumferential groove 88 about the longitudinal axis A opening radially opposite the longitudinal axis A.

As can be seen in FIG. 3, the longitudinal segment 78 comprises, at its distal end 82, a flared portion 89A that has an outer chamfer 89B. The circumferential groove 88 is defined in the flared portion 89A of the longitudinal segment 78, between the outer chamfer 89B and the head for mounting 84.

The circumferential groove 88 receives the free edge 60 of each contact tongue 58 of the gripping member 16.

The contact tongues 58 facilitate the assembly of the rod carrier 18 with the gripping member 16, via their snap-fitting into the circumferential groove 88. This assembly is easily obtained by relative translation along the axis A of the gripping member 16 with respect to the rod carrier 18, generating an axial deformation of the contact tongues 56. The cooperation between the inner chamfer on the free end 60 of each contact tongue 58 and the outer chamfer 89B facilitates the snap-fitting.

The longitudinal extend of the groove 88, taken along the axis A, is greater than the longitudinal extent of the free edge 60, taken along the same axis A by at least 10%.

As such, the rod carrier 18 has, on the free edge 60 of each contact tongue 58, a second linear contact region 90 with the gripping member 16. The second linear contact region 90 is a circumferential linear contact region.

In the example shown, the contact tongues 58 are discontinuous in the circumferential direction by being separated from each other by the slots 56. The second linear contact region 90 of the rod carrier 18 is as such also continuous in the circumferential direction. Alternatively, the rod carrier 18 has a contact tongue 58 that is continuous in the circumferential direction forming a continuous annular flange, made of a flexible material in order to allow for the snap-fitting of the rod carrier or associated with a washer for blocking in translation along the axis A integral with the rod carrier 18. Those skilled in the art will be able to determine the material to be chosen for the contact tongue 58 in order to have sufficient flexibility to allow for the snap-fitting of the rod carrier 18, according to the material chosen for the latter. The second linear contact region 90 of the rod carrier 18 is as such also continuous in the circumferential direction.

The first linear contact region 86 and the second linear contact region 90 are separated by a minimum distance I. The minimum distance I is, for example, greater than 20 mm and is preferably between 23 mm and 60 mm.

The minimum distance I is greater than or equal to two times the average diameter between the diameter D1 of the rod carrier 18 in the first linear contact region 86 and the diameter D2 of the rod carrier 18 in the second linear contact region 90.

The first linear contact region 86 has a contact length 11 taken along the longitudinal axis A less than 5%, preferably less than 2% of the minimum distance I separating along the axis A the first linear contact region 86 and the second linear contact region 90.

Advantageously, the contact length 11 is less than 5%, preferably less than 2% of the diameter D1 of the longitudinal segment 78 taken on the first contact region 86.

The contact length 11 is as such less than 1 mm, and preferably between 0.05 mm and 0.5 mm.

Likewise, the second linear contact region 90 has a contact length 12 taken along the longitudinal axis A less than 5%, preferably less than 2% of the minimum distance I separating along the axis A the first linear contact region 86 and the second linear contact region 90.

On the second contact region 90, the circumferential groove 88 has a maximum diameter D2. The maximum diameter D2 is, for example, between 5.50 mm and 6 mm.

Advantageously, the contact length 12 is less than 5%, preferably less than 2% of the maximum diameter D2 of the circumferential groove 88 taken on the second contact region 90.

The contact length 12 is as such less than 1 mm, and preferably between 0.05 mm and 0.5 mm.

Advantageously, the contact regions 86, 90 are adapted so that, when a mechanical stress, resulting in a motion with respect to the longitudinal axis A, is applied on the first application member 22A, a rotation of the rod 20 and of the rod carrier 18 with respect to the gripping member 16 is obtained as soon as the motion crosses a limit value.

The longitudinal segment 78 has an intermediate region 92, shown in FIG. 1, defined between the first linear contact region 86 and the second linear contact region 90.

In the intermediate region 92, the rod carrier 18 is arranged entirely separated from the gripping member 16. More particularly, in the intermediate region 92, the rod carrier 18 and the gripping member 16 define a radial gap, measured perpendicularly to the longitudinal axis A, at least equal to 5%, preferably at least equal to 10%, and more preferably at least equal to 50% of the maximum thickness presented by the rod carrier 18, over at least 50% of the length of the intermediate region 92 taken along the longitudinal axis A.

The rod carrier 18 and the gripping member 16 have a functional radial gap between them in the first circumferential linear contact region 86 and/or in the second circumferential linear contact region 90.

Preferably, this functional radial gap is less than 0.5 mm, and more preferably less than 0.2 mm and in particular in the neighborhood of 0.1 mm. As such, when the rod carrier 18 and the gripping member 16 are placed coaxially to one another, such as shown in FIGS. 1 to 3, a gap in the radial direction exists on either side of the rod carrier 18, between the rod carrier 18 and the gripping member 16, in the first circumferential linear contact region 86 and/or in the second circumferential linear contact region 90, with this gap substantially corresponding to half of the aforementioned functional radial gap.

Advantageously, in the intermediate region 92, the rod carrier 18 and the gripping member 16 define a radial gap, i.e. a gap in the radial direction, greater than or equal to 1.1 times, preferably 1.5 times, the functional radial gap defined between the rod carrier 18 and the gripping member 16 in the first circumferential linear contact region 86 and/or in the second circumferential linear contact region 90, over at least 50% of the length of the intermediate region 92 taken along the longitudinal axis A. The term “radial gap” in the intermediate region 92 between the rod carrier 18 and the gripping member 16 here means the distance in the radial direction that separates the rod carrier 18 and the gripping member 16 on the intermediate region 92, when these two elements are arranged coaxially. As such, if the functional radial gap between the rod carrier 18 and the gripping member 16 in the first circumferential linear contact region 86 and/or in the second circumferential linear contact region 90 is for example 0.1 mm, then the radial gap between the rod carrier 18 and the gripping member 16 in the intermediate region 92, on one side and the other of the longitudinal axis A when the rod carrier 18 and the gripping member 16 are arranged coaxially, is greater than or equal to 0.11 mm, and preferably greater than or equal to 0.15 mm.

In reference to FIG. 3, the head for mounting 84 of the rod carrier 18 comprises a central ring 93 for fastening the rod 20 and a connecting skirt 94 protruding around the central ring 93. The connecting skirt 94 extends to the distal edge 40 of the cover 34, from the distal end 82 of the longitudinal segment 78.

The inner surface 96 of the connecting ring 94 is threaded, and is able to cooperate with the threading present on the neck 30 of the receptacle 12.

The connecting skirt 94 also comprises a longitudinal protrusion 100, that can be seen in FIG. 5, able to cooperate with a stop 102 of the receptacle 12, which can be seen in FIG. 3, in order to ensure the blocking in rotation of the rod carrier 18 with respect to the receptacle 12 in idle position.

The ring 94 and the longitudinal segment 78 define a central housing 104. In the example of FIG. 3, the central housing 104 extends to the proximal end 80 of the rod carrier 18 until a bottom 106, arranged longitudinally beyond the circumferential groove 88 of the longitudinal segment 78.

The blocking device 19 is configured to limit the relative translation along the axis A of the rod carrier with respect to the gripping member to a functional gap between these elements. This functional gap is preferably less than 2 mm, in particular less than 1 mm and in particular in the neighborhood of 0.7 mm, even in the neighborhood of 0.5 mm.

The blocking device 19 comprises a first axial stop surface 108A formed on the rod carrier 18, and a second axial stop surface 108B formed by the gripping member 16.

Preferably, the first and second axial stop surfaces 108A, 108B are adjacent along the axis A at one at least of the first and second contact regions 86, 90.

In the example in FIG. 3, they are adjacent to the second contact region 90. The first axial stop surface 108A is as such formed by a shoulder defined in the flared portion 89A by the circumferential groove 88, and the second stop surface 108B is formed by a proximal surface of the transverse wall 54 of the cover 34, on the free edge 60.

The rod 20 is added in the central housing 104.

The system for the selective blocking in rotation 24 of the rod carrier 18 with respect to the gripping member 16 is shown in FIGS. 5 to 7.

The blocking system 24 can be maneuvered between an inactive configuration, in which the rod carrier 18 is freely rotating about the longitudinal axis A, and an active configuration, in which the rod carrier 18 is entirely blocked in rotation with respect to the gripping member 16. The term total blocking here means a “frank” contact of elements abutting against one another, and not only a friction contact.

The blocking system 24 comprises splines 112 formed on the rod carrier 18, and at least one flexible tab 114 able to cooperate with the splines 112. It comprises, for each flexible tab 114, a flexible hinge 116 for connecting the flexible tab 114 to the cover 34 of the gripping member 16.

The or each flexible tab 114, the flexible hinge 116 and the cover 34 are advantageously from the same material.

The splines 112, which can be seen in FIG. 5, are arranged on an external peripheral surface of the rod carrier 18. In particular, the splines 112 are arranged on an outer surface 98 of the connecting ring 94 of the rod carrier 18.

In the example of FIGS. 6 and 7, the blocking system 24 comprises two flexible tabs 114 diametrically opposite with respect to the axis A.

Each flexible tab 114 is mounted mobile on the gripping member 16.

It is defined by a cut made through the gripping member 16. The cut is here of a rounded shape, for example in the shape of a C.

Each flexible tab 114 comprises at least one locking cog 118 radially protruding towards the axis A. The locking cog 118 is able to be inserted into a spline 112 in the active configuration of blocking, in order to block the rod carrier 18 in rotation with respect to the gripping member 16. It is arranged entirely apart from the splines 112 in the deactivated configuration.

The first application member 22A is also designated by the term “brush” in what follows. Preferably, the first application member 22A is made of elastomer.

The first application member 22A is fixed to the free end of the rod 20.

It is able to be driven in rotation by keratin fibers, in particular about the longitudinal axis A, with respect to the gripping member 16.

As shown in FIGS. 8 and 9, the first application member 22A comprises a support body 120 and a plurality of pins 122 formed on an outer surface 124 of the support body 120, the pins 122 being formed on the outer surface 124 in the form of rows.

The first application member 22A advantageously has an axial symmetry along the longitudinal axis A.

The first application member 22A comprises an even number of rows of pins.

The number of rows of pins is here equal to 12. Alternatively, the number of rows of pins is greater than or equal to 8, and advantageously between 8 and 16.

The support body 120 has a substantially circular section transversal to the longitudinal axis A.

The transversal section of the support body 120 has a diameter between 1.5 mm and 4 mm.

The support body 120 is made of elastomer. Alternatively, the support body 120 is for example made of hytrel, SEBS or polypropylene (PP).

Each pin 122 has a substantially conical shape of revolution.

On the outer surface 124 of the support body 120, each pin 122 has a section with a diameter between 0.1 mm and 1 mm.

The pins 122 of the same row have the same section taken on the outer surface 124 of the support body 120. More generally, all of the pins 122 of the first application member 22A have the same section taken on the outer surface 124 of the support body 120.

Each pin 122 has a height greater than 2 mm, advantageously between 1.5 mm and 5 mm.

The pins 122 of the same row have an identical height. More generally, all of the pins 122 of the first application member 22A have an identical height.

Each pin 122 extends from the outer surface 124 along a predetermined direction perpendicular to the longitudinal axis A. The predetermined direction of a pin is a straight line passing through the middle of any section of said pin parallel to the longitudinal axis A.

The pins 122 of the same row have predetermined parallel directions.

Each pin 122 is as a single piece with the support body 120, each pin 122 is then made of elastomer. Alternatively at least one portion of the pins 122 is made from a material different from that of the support body 120 by overmolding on the support body 120.

Each row of pins extends parallel to the longitudinal axis A.

Each row of pins has a length greater than 2 mm.

Each row of pins comprises a number of pins 122 greater than 20 and advantageously between 30 and 50.

The difference between two adjacent pins of the same row is identical for all of the pins of said row. Two pins of the same row are considered to be adjacent if no pin of said row is arranged between these two pins.

The difference between two adjacent pins of the same row is between 1% and 10% of the height of the pins 122.

The difference between two adjacent pins of the same row is identical for all of the rows of the first application member 22A.

A first row 126 of pins is arranged adjacent to a second row 128 of pins, and a third row 130 of pins is arranged adjacent to the second row 128 of pins. Two rows of pins are considered to be adjacent if no row of pins is arranged between these two rows.

As shown in FIG. 8, preferably, the second row 128 of pins comprises one pin 122 less than the first row 126 of pins. The first row 126 of pins and the third row 130 of pins comprise the same number of pins 122. They comprise here 21 pins.

More generally, two rows of the first application member 22A separated by a single row of pins comprise the same number of pins 122.

In particular, the second row 128 comprises a number of pins equal to the number of pins 122 of the rows adjacent to the first row 126 and to the third row 130. These rows here comprise 20 pins.

Preferably, according to a first embodiment, at least one pin of the second row 128 is arranged axially along the longitudinal axis A between two adjacent pins of the first row 126.

In particular, all of the pins 122 of the first row 126 are axially offset along the longitudinal axis A relative to the pins 122 of the second row 128 by a predetermined distance taken on the outer surface 124 of the support body 120.

The predetermined distance is greater than 1% of the height of the pins 122.

In the example shown in FIG. 8, each pin of the second row 128 is located midway axially along the longitudinal axis A of two adjacent pins 122 of the first row 126.

In other terms, the predetermined distance is substantially equal to half the sum of the diameter of a pin and of the difference between two adjacent pins 122, said difference and said diameter being taken on the outer surface 124 of the support body 120.

The predetermined directions of the pins 122 of the second row 128 are contained in the angular sector defined by the predetermined directions of the pins 122 of the first row 126 and the predetermined directions of the pins 122 of the third row 130.

Each pin 122 of the first row 126 is axially positioned, along the longitudinal axis A, at the same position as a pin of the third row 130. In other terms, the third row 130 is not axially offset with respect to the first row 126.

More generally, two rows of the first application member 22A separated by a single row of pins 122 are not axially offset with respect to one another.

In particular, each pin of the second row 128 is as such axially positioned, along the longitudinal axis A, at the same position as a pin of the rows adjacent to the first row 126 and to the third row 130.

The distance, taken on the outer surface 124, between a pin of the first row 126 and at least one pin of the third row 130 is less than 30% of the height of the pins 122.

As shown in FIG. 9, the distance between a pin of the first row 126 and the pin of the third row 130 axially positioned at the same position is less than the diameter of the section of the pins 122, said distance and said section being taken on the outer surface 124.

In projection in a plane perpendicular to the longitudinal axis (A), each pin of the second row 128 of pins is arranged between pins of the first row 126 and pins on the third row 130, preferably in the middle of the pins of the first row 126 and pins of the third row 130.

More precisely, in projection in a plane perpendicular to the longitudinal axis A, the predetermined direction of each pin of the second row 128 is the bisector of the angle formed between the predetermined direction of a pin of the first row 126 and the predetermined direction of a pin of the third row 130.

In other words, each pin of the second row 128 is arranged staggered with two adjacent pins of the first row 126 and two adjacent pin of the third row 130.

More generally, each pin of a row that has one pin less than its adjacent rows, is arranged staggered with two adjacent pins of one of its rows and two adjacent pins of the other of its adjacent rows.

Even more generally, even if the structure of the rod carrier 18 is different from that shown in the figures, the rod carrier 18 has at least two linear contact regions with the gripping member providing a free rotation and guided with respect to the gripping member.

Preferably, the requisite according to the invention presents one or more of the following features:
it comprises an applicator 14 comprising the gripping member 16, extending along the longitudinal axis A, a rod carrier 18, arranged in the gripping member 16 and mounted freely mobile in rotation about the longitudinal axis A with respect to the gripping member 16, a rod 20 secured to the rod carrier 18 and carrying the application member 22A, 22B, wherein the rod carrier 18 as such has a first linear contact region 86 with the gripping member 16, said first linear contact region 86 being a circumferential linear contact region about the longitudinal axis A, and a second linear contact region 90 with the gripping member 16, said second linear contact region 90 being a circumferential linear contact region;
the first linear contact region 86 and the second linear contact region 90 are separated by a minimum distance I, which is greater than or equal to two times the average diameter between the diameter D1 of the rod carrier 18 in the first linear contact region 86 and the diameter D2 of the rod carrier 18 in the second linear contact region 90;
each first and second linear contact region 86, 90 has a contact length 11, 12 taken along the longitudinal axis A less than 5%, preferably less than 2% of the minimum distance I separating along the axis A the first linear contact region 86 and the second linear contact region 90;
the gripping member 16 comprises an inner sleeve 66, delimiting a cavity 74 in which the rod carrier 18 is received, the inner sleeve 66 comprising a bulge 76, the first linear contact region 86 extending opposite the bulge 76;
the gripping member 16 comprises a peripheral wall 38 about the longitudinal axis A and at least one contact tongue 58 radially protruding from the peripheral wall 38, the or each contact tongue 58 defining a free inner edge directed towards the longitudinal axis A, the second linear contact region 90 extending opposite the free edge 60;
the rod carrier 18 defines a circumferential groove 88 that receives the free edge 60 of the or each contact tongue 58;
the gripping member 16 comprises a transverse wall 54 that defines a plurality of radial slots 56, the radial slots 56 delimiting between them at least two contact tongues 58 located facing the second linear contact region 90;
the gripping member 16 comprises a plug 36 and a cover 34, the plug 36 being attached to the cover 34,

the first linear contact region 86 being delimited opposite the plug 36,

the second linear contact region 90 being delimited opposite the cover 34;
the rod carrier 18 is locked along any rotation following an axis perpendicular to the longitudinal axis A of the gripping member 16.

Makeup Removal Composition for Keratin Fibers

The makeup removal composition, according to the invention, comprises a physiologically acceptable medium, i.e. a non-toxic medium and able to be applied on keratin fibers, in particular the eyelashes, in particular compatible with the eye area.

### Aqueous Phase

The makeup removal composition according to the invention comprises an aqueous phase. It also preferably comprises at least 30% by weight of water with respect to the total weight of the composition.

Preferably, the makeup removal composition according to the invention comprises a continuous aqueous phase: The term “continuous” aqueous phase means that the composition has a conductivity, measured at 25° C., greater than 23 μS/cm (microSiemens/cm), with the conductivity being measured for example using a Mettler Toledo MPC227 conductometer and an Inlab730 conductivity measuring cell. The measuring cell is immersed in the composition, in such a way as to remove air bubbles that can form between the two electrodes of the cell. Reading the conductivity is done as soon as the value of the conductometer has stabilized. An average is taken over at least three successive measurements.

The aqueous phase can be comprised primarily of water; it can also comprise a mixture of water and water-miscible solvent (miscibility in water greater than 50% by weight at 25° C.) such as lower monoalcohols having from 1 to 5 carbon atoms such as ethanol, isopropanol, glycols having from 2 to 8 carbon atoms such as propylene glycol, caprylyl glycol, 1,3-butylene glycol, hexylene glycol, dipropylene glycol, C3-C4 ketones, C2-C4 aldehydes and mixtures thereof.

Preferably, the aqueous phase (water and possibly the water-miscible solvent) is present in a content at least equal to 40% by weight with respect to the total weight of the composition, preferably at least equal to 43% by weight, preferably at least equal to 45% by weight.

Preferably, the aqueous phase (water and possibly the water-miscible solvent) is present in a content between 40% and 65% by weight of the total weight of the composition, preferably between 43% and 60% by weight, preferably between 45% and 60% by weight. Preferably, the aqueous phase comprises at least 40% by weight of water with respect to the total weight of the composition.

### Surfactants

The makeup removal compositions according to the invention also comprise at least one surfactant.

This surfactant can be present in particular in a proportion ranging from 0.1% to 20%, and better from 0.3% to 15% by weight with respect to the total weight of the composition.

According to the invention, an emulsifier suitable chosen for obtaining an oil-in-water emulsion is generally used. In particular, an emulsifier can be used that has at 25° C. an HLB balance (hydrophilic-lipophilic balance) in terms of GRIFFIN, preferably greater than or equal to 8.

The HLB value as per GRIFFIN is defined in J. Soc. Cosm. Chem. 1954 (volume 5), pages 249-256.

These surfactants may be chosen from among anionic, cationic, amphoteric and non-ionic surfactants. Reference may be made to the document “Encyclopedia of Chemical Technology, KIRK-OTHMER”, volume 22, p. 333-432, 3rd edition, 1979, WILEY, for the definition of the properties and (emulsifying) functions of surfactant agents, in particular p. 347-377 of this reference, for anionic, amphoteric and non-ionic surfactants.

Preferably, the surfactant according to the invention is chosen from:
a) anionic surfactants such as:
   polyoxyethylenated fatty acid salts and particularly those derived from alkaline salts, and mixtures thereof;
   phosphoric esters and their salts such as “DEA oleth-10 phosphate” (Crodafos N 10N from CRODA) or monopotassium monocetyl phosphate (Amphisol K from Givaudan);
   sulfosuccinates such as “Disodium PEG-5 citrate lauryl sulfosuccinate” and “Disodium ricinoleamido MEA sulfosuccinate”;
   alkylethersulfates such as sodium lauryl ether sulfate (INCI name: sodium laureth sulfate), magnesium lauryl ether sulfate (INCI name: magnesium laureth sulfate), sodium oleyl ether sulfate (INCI name: sodium oleth sulfate), magnesium oleyl ether sulfate (INCI name: magnesium oleth sulfate);
   isethionates;
   acylglutamates such as “Disodium hydrogenated tallow glutamate” (AMISOFT HS-21 R® marketed by AJINOMOTO) and sodium stearoyl glutamate (AMISOFT HS-11 PF® marketed by AJINOMOTO) and mixtures thereof;
   derivatives of soybeans such as potassium soyate;
   citrates, such as Glyceryl stearate citrate (Axol C 62 Pellets from Degussa);
   derivatives of proline, such as Sodium palmitoyl proline (Sepicalm VG from Seppic), or the Mixture of Sodium palmitoyl sarcosinate, Magnesium palmitoyl glutamate, palmitic acid and Palmitoyl proline (Sepifeel One from Seppic);
   lactylates, such as Sodium stearoyl lactylate (Akoline SL from Karlshamns AB);
   sarcosinates, such as sodium palmitoyl sarcosinate (Nikkol sarcosinate PN) or the mixture of Stearoyl sarcosine and Myristoyl sarcosine 75/25 (Crodasin SM from Croda);
   sulfonates, such as Sodium C14-17 alkyl sec sulfonate (Hostapur SAS 60 from Clariant);
   glycinates, such as sodium cocoyl glycinate (Amilite GCS-12 from Ajinomoto).
   C16-C30 fatty acid salts in particular those derived from amines, such as triethanolamine stearate and/or amino-2-methyl-2-propane di-ol-1,3 stearate;
b) amphoteric surfactants, such as N-acyl-aminoacids such as N-alkyl-aminoacetates, disodium cocoamphodiacetate and amine oxides such as stearamine oxide or even silicone surfactants such as dimethicone copolyol phosphates such as the one sold under the trade name PECOSIL PS 1000 by PHOENIX CHEMICAL;
c) non-ionic surfactants, such as:
   esters and ethers of oses such as the mixture of cetylstearyl glucoside and cetyl and stearyl alcohols such as Montanov 68 from Seppic;
   oxyethylene and/or oxypropylene ethers (that may comprise from 1 to 150 oxyethylene and/or oxypropylene groups) of glycerol;
   oxyethylene and/or oxypropylene ethers (that may comprise from 1 to 150 oxyethylene and/or oxypropylene groups) of fatty alcohols (particularly C8-C24 and preferably C12-C18 alcohols) such as oxyethylene ether of cetearylic alcohol with 30 oxyethylene groups (CTFA name “Ceteareth-30”), oxyethylene ether of stearylic alcohol with 20 oxyethylene groups (CTFA name “Steareth-20”), and oxyethylene ether of the mix of C12-C15 fatty alcohols containing 7 oxyethylene groups (CTFA name “C12-15 Pareth-7”) marketed under the name NEODOL 25-7® by SHELL CHEMICALS,
   fatty acid esters (in particular C8-C24 acid, and preferably C16-C22) and polyethylene glycol (able to comprise from 1 to 150 ethyleneglycol patterns) such as PEG-50 stearate and PEG-40 monostearate sold under the trade name MYRJ 52P® by ICI UNIQUEMA,
   fatty acid esters (particularly C8-C24 acid, and preferably C16-C22 acid) and oxyethylenated and/or oxypropylated glycerol ethers (that may include 1 to 150 oxyethylanated and/or oxypropylenated groups), such as PEG-200 glyceryl monostearate sold particularly under the name Simulsol 220 TM® by SEPPIC; polyethoxylated glyceryl stearate with 30 ethylene oxide groups such as the TAGAT S® product sold by GOLDSCHMIDT, polyethoxylated glyceryl oleate with 30 ethylene oxide groups like the TAGAT O® product sold by GOLDSCHMIDT, polyethoxylated glyceryl cocoate with 30 ethylene oxide groups like the VARIONIC LI 130 product sold by SHEREX, polyethoxylated glyceryl isostearate with 30 ethylene oxide groups such as the TAGAT L® product sold by GOLDSCHMIDT and polyethoxylated glyceryl laurate with 30 groups of ethylene oxide like the TAGAT I® product from GOLDSCHMIDT,
   fatty acid esters (particularly C8-C24 acid and preferably C16-C22 acid) and oxyethylenated and/or oxypropylenated sorbitol ethers (possibly containing 1 to 150 oxyethylenated and/or oxypropylenated groups), such as polysorbate 20 sold under the name Tween 20® by CRODA, polysorbate 60 sold under the name Tween 60® by CRODA,
   polydimethylsiloxanes comprise both oxyethylenated groups and oxypropylene groups, such as dimethicone copolyol with INCI name PEG/PPG-17/18 DIMETHICONE, such as that sold under the trade name Q2-5220 Resin Modifier® by DOW CORNING,
   dimethicone copolyol benzoate (FINSOLV SLB 101® and 201® from FINTEX),
   copolymers of propylene oxide and of ethylene oxide (also called EO/PO polycondensates), and more particularly copolymers consisting of polyethylene glycol/polypropylene glycol blocks, such as for example polyethylene glycol/polypropylene glycol/polyethylene glycol triblock polycondensates, for example those having the following chemical structure:

H—(O—CH2-CH2)a-(O—CH(CH3)-CH2)b-(O—CH2-CH2)a-OH,
in which formula a ranges from 2 to 120, and b ranges from 1 to 100. As a EO/PO polycondensate that can be used, mention can be made of polyethylene glycol/polypropylene glycol/polyethylene glycol triblock polycondensates sold under the trade names SYNPERONIC® such as SYNPERONIC PE/L44® and SYNPERONIC PE/F127® by ICI; and
d) mixtures thereof.

Preferably the makeup removal composition comprises at least one anionic surfactant, preferably a mixture of at least two anionic surfactants, and/or at least one amphoteric surfactant.

Preferably the makeup removal composition comprises the mixture of the following surfactants:
at least one anionic surfactant chosen from alkylethersulfates such as sodium lauryl ether sulfate, magnesium lauryl ether sulfate, sodium oleyl ether sulfate or magnesium oleyl ether sulfate; and
at least one amphoteric surfactant, preferably disodium cocoamphodiacetate.

### Gelling Agents

The makeup removal composition according to the invention can comprise a gelling agent of the aqueous phase.

The gelling agents of the aqueous phase that can be used in the makeup removal compositions of the invention can be chosen from:
homo- or copolymers of acrylic or methacrylic acids or the salts thereof and the esters thereof and in particular the products sold under the trade names VERSICOL F® or VERSICOL K® by the company ALLIED COLLOID, UTRAHOLD 8® by the company CIBA-GEIGY, polyacrylic acids of the SYNTHALEN K type,
acrylic and acrylamide acid copolymers sold in the form of their sodium salt under the names RETEN by HERCULES, sodium polymethacrylate sold under the name DARVAN No 7 by VANDERBILT, the sodium salts of polyhydroxycarboxylic acids sold under the name HYDAGEN F by HENKEL,
polyacrylic acid/alkyl acrylate copolymers of the PEMULEN type,
AMPS (Polyacrylamidomethyl propane sulfonic acid partially neutralized with ammonia and highly cross-linked) marketed by the company CLARIANT, as well as AMPS/acrylamide copolymers of the SEPIGEL® or SIMULGEL® type marketed by the company SEPPIC, and the AMPS/polyoxyethylenated alkyl methacrylate (cross-linked or not),
cellulose polymers such as hydroxyethylcellulose, hydroxypropylcellulose, methylcellulose, ethylhydroxyethylcellulose, carboxymethylcellulose, and quaternized cellulose derivatives;
vinyl polymers, such as polyvinylpyrrolidones, methylvinyl ether and malic anhydride, vinyl acetate and crotonic acid copolymer, vinylpyrrolidone and vinyl acetate copolymers; vinylpyrrolidone and caprolactam copolymers; polyvinyl alcohol;
polymers of natural origin, possibly modified, such as guar gum, xanthan derivatives and carrageenans;
and mixtures thereof.

The gelling agent can be present in the makeup removal composition according to the invention with a dry matter content ranging from 0.4% to 10% by weight, preferably from 0.5% to 8% by weight and better from 0.5% to 5% by weight in relation to the total weight of the composition.

### Oily Phase

The makeup removal composition according to the invention can comprise an oily phase. This oily phase can be dispersed.

Preferably, the oily phase is present in a content ranging from 0.1% to 50% by weight, preferably from 1% to 30% by weight relative to the total weight of the composition.

Preferably, the oily phase comprises at least one liquid non-aqueous oil or fatty body at ambient temperature (25° C.) and atmospheric pressure (760 mm of Hg).

The oil can be chosen from volatile oils and/or non-volatile oils, and mixtures thereof.

The oil or oils may be present in the makeup removal composition in a content ranging from 0.5% to 50% by weight, preferably from 5% to 30% by weight relative to the total weight of the composition.

The term “volatile oil” is intended to mean any oil capable of evaporating on contact with keratin fibers, in less than one hour, at ambient temperature and at atmospheric pressure. The volatile organic solvent(s) and volatile oils according to the invention are organic solvents and volatile cosmetic oils, which are liquid at ambient temperature, having a vapor pressure different to zero, at ambient temperature and atmospheric pressure, particularly ranging from 0.13 Pa to 40,000 Pa (10⁻³ at 300 mm Hg), particularly ranging from 1.3 Pa to 13,000 Pa (0.01 to 100 mm Hg), and more specifically ranging from 1.3 Pa to 1300 Pa (0.01 to 10 mm Hg). The term “non-volatile oil” denotes an oil remaining on the keratin fiber at ambient temperature and atmospheric pressure for at least several hours and particularly having a vapor pressure less than 10⁻³ mm Hg (0.13 Pa).

These oils may be hydrocarbon oils, silicone oils, fluorinated oils or mixtures thereof.

A “hydrocarbon oil” is an oil containing principally hydrogen and carbon atoms and possible oxygen, nitrogen, sulfur and phosphorus atoms. The volatile hydrocarbon oils can be chosen from hydrocarbon oils having 8 to 16 carbon atoms, and particularly branched C8-C16 alkanes such as petroleum-based C8-C16 isoalkanes (also referred to as isoparaffins) such as isododecane (also referred to as 2,2,4,4,6-pentamethylheptane), isodecane, isohexadecane, and for example oils sold under the trade names Isopars or Permetyls, branched C8-C16 esters, isohexyl neopentanoate, and mixtures thereof. Further volatile hydrocarbon oils such as petroleum distillates, particularly those sold under the name Shell Solt by SHELL, may also be used. Preferably, the volatile solvent is chosen from volatile hydrocarbon oils having 8 to 16 carbon atoms and mixtures thereof. Other volatile oils that can be used are volatile silicones, such as for example volatile linear or cyclic silicone oils, particularly those having a viscosity ≤8 centistokes (8 10⁻⁶ m2/s), and in particular having 2 to 7 silicon atoms, these silicones optionally comprising alkyl or alkoxy groups having 1 to 10 carbon atoms. Mention may be made, as a volatile silicone oil suitable for use in the invention, in particular, of octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof. Mention may also be made of volatile alkyl trisiloxane oils with general formula (I):

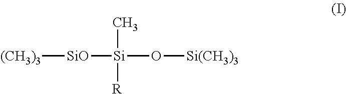

In which R represents an alkyl group comprising 2 to 4 carbon atoms and in which one or several hydrogen atoms can be substituted by a fluorine or chlorine atom. Among oils with general formula (I), mention may be made of:
3-butyl 1,1,1,3,5,5,5-heptamethyl trisiloxane,
3-propyl 1,1,1,3,5,5,5-heptamethyl trisiloxane, and
3-ethyl 1,1,1,3,5,5,5-heptamethyl trisiloxane,
   corresponding to the oils having formula (I) for which R is respectively a butyl group, a propyl group or an ethyl group.

The makeup removal composition can also comprise at least one non-volatile oil, and in particular chosen from non-volatile hydrocarbon and/or silicone and/or fluorinated oils. As a non-volatile hydrocarbon oil, mention may be made of:
hydrocarbon oils of plant origin such as triesters of fatty acids and glycerol for which the fatty acids can have chain lengths ranging from C4 to C24, with the latter able to be linear or branched, saturated or unsaturated; these oils are in particular wheat germ, sunflower, grape seed, sesame, corn, apricot, castor, shea, avocado, olive, soybean oils, sweet almond, palm, rapeseed, cotton, hazelnut, macadamia, jojoba, alfalfa, poppy seed, pumpkin, sesame, squash, rapeseed, blackcurrant, evening primrose, millet, barley, quinoa, rye, safflower, candlenut, passiflora, musk rose oil; or caprylic/capric acid triglycerides such as those sold by Stearineries Dubois or those sold under the trade names Miglyol 810, 812 and 818 by Dynamit Nobel;
linear or branched hydrocarbons of mineral or synthetic origin, such as liquid paraffins and derivatives thereof, polydecenes, polybutenes, hydrogenated polyisobutene such as Parleam, squalane;
synthetic ethers having from 10 to 40 carbon atoms;
synthetic esters such as the oils having the formula R1COOR2 wherein R1 represents the residue of a linear or branched fatty acid comprising 1 to 40 carbon atoms and R2 represents a hydrocarbon chain, particularly branched containing 1 to 40 carbon atoms where R1+R2≥10, such as for example Purcellin oil (cetostearyl octanoate), isopropyl myristate, isopropyl palmitate, C12 to C15 alcohol benzoate, hexyl laurate, diisopropyl adipate, isononyl isononanoate, 2-ethylhexyl palmitate, isostearate of isostearate, alcohol or polyalcohol octanoates, decanoates or ricinoleates such as propylene glycol dioctanoate; hydroxylated esters, such as isostearyl lactate, diisostearyl malate; and pentaerythritol esters;
fatty alcohols that are liquid at ambient temperature, with a branched and/or unsaturated carbon chain having 12 to 26 carbon atoms, such as octyldodecanol, isostearyl alcohol, oleic alcohol, 2-hexyldecanol, 2-butyloctanol, 2-undecylpentadecanol;
higher fatty acids, such as oleic acid, linoleic acid, linolenic acid;
and mixtures thereof.

The non-volatile silicone oils that can be used in the makeup removal composition in accordance with the invention can be non-volatile polydimethylsiloxanes (PDMS), polydimethylsiloxanes comprising alkyl or alkoxy groups which are pendant or at the end of the silicone chain, groups each having 2 to 24 carbon atoms; phenylated silicones, such as phenyl trimethicones, phenyl dimethicones, phenyltrimethylsiloxydiphenylsiloxanes, diphenyl dimethicones, diphenylmethyldiphenyl-trisiloxanes or (2-phenylethyl)trimethylsiloxysilicates.

The fluorinated oils that can be used in the invention are in particular fluorosilicone oils, fluorinated polyethers, fluorinated silicones, as described in document EP-A-847752.

The makeup removal composition according to the invention can be manufactured by known methods, generally used in the cosmetics field.

Coating Composition for Keratin Fibers

As indicated hereinabove, the cosmetic application requisite according to the invention can comprise a coating composition for keratin fibers.

Such a coating composition comprises a physiologically acceptable medium, i.e. a non-toxic medium and able to be applied on keratin fibers, in particular the eyelashes, in particular compatible with the eye area.

The coating composition is preferably a mascara.

The term “mascara” denotes a composition intended to be applied on the eyelashes: it can be a makeup composition for the eyelashes, a makeup base for eyelashes or base-coat, a composition to be applied on a mascara, also called a top-coat, or a composition for the cosmetic treatment of eyelashes.

Preferably, the coating composition comprises at least one an aqueous phase, such as described for the makeup removal composition. It also preferably comprises at least 30% by weight of water with respect to the total weight of the composition.

The coating composition can further comprise at least one surfactant and/or at least one gelling agent of the aqueous phase such as mentioned hereinabove.

Oily Phase

The coating composition preferably comprises an oily phase, preferably a dispersed oily phase. The fatty phase can be present in a content between 5% and 60% by weight, preferably between 10% and 40% by weight with respect to the total weight of the composition.

The fatty phase can comprise at least one wax, at least one oil and/or at least one pasty fatty substance. The oil can be a volatile or non-volatile oil such as mentioned hereinabove.

Preferably, the coating composition comprises at least one wax.

The term “wax” refers to a lipophilic compound, which is solid at ambient temperature (25° C.), deformable or not, having a reversible solid/liquid change of state and a melting point greater than or equal to 40° C. that can range up to 120° C. In particular, the waxes suitable for the invention may have a melting point greater than or equal to 45° C., and particularly greater than or equal to 55° C.

The term “lipophilic compound” compound means a compound that has an acid index and a hydroxyl index less than 150 mg KOH/g.

According to the invention, the melting point is equivalent to the temperature of the most endothermic peak observed in thermal analysis (DSC) as described in the standard ISO 11357-3; 1999. The melting point of the wax may be measured using a differential scanning calorimeter (DSC), for example the calorimeter sold under the name “MDSC 2920” by TA Instruments.

The measurement protocol is as follows:

A 5 mg sample of wax placed in a crucible is subjected to a first temperature rise from −20° C. to 100° C., at a heating rate of 10° C./minute, and is then cooled from 100° C. to −20° C. at a cooling rate of 10° C./minute and finally subjected to a second temperature rise from −20° C. to 100° C. at a heating rate of 5° C./minute. During the second temperature rise, the variation in the difference in power absorbed by the empty crucible and by the crucible containing the wax sample as a function of temperature is measured. The melting point of the compound is the value of the temperature equivalent to the top point of the peak of the curve representing the variation in the difference in power absorbed as a function of temperature.

The waxes may be hydrocarbon, silicone and/or fluorinated and be of plant, mineral, animal and/or synthetic origin.

The wax may be present in a content ranging from 1% to 30% by weight with respect to the total weight of the composition, better from 2% to 20%, and even better from 5% to 15% by weight.

Hydrocarbon waxes such as beeswax, lanolin wax; rice wax, Carnauba wax, Candellila wax, Ouricury wax, Japan wax, Berry wax, shellac wax and sumac wax; montan wax can be in particular used as wax.

According to an embodiment, a hydrocarbon wax will be used chosen from beeswax, rice bran wax, Carnauba wax, and mixtures thereof.

Mention may also be made of waxes obtained by means of the catalytic hydrogenation of animal or plant oils having C8-C32 linear or branched fat chains.

Of these, particular mention may be made of hydrogenated jojoba oil, hydrogenated palm oil, hydrogenated sunflower oil, hydrogenated castor oil, hydrogenated coconut oil, hydrogenated lanolin oil, di-(trimethylol-1,1,1 propane) tetrastearate sold under the name “HEST 2T-4S” by HETERENE, di-(trimethylol-1,1,1 propane) tetrabehenate sold under the name HEST 2T-4B by HETERENE.

The wax used can also be obtained by hydrogenating esterified olive oil with stearyl alcohol sold under the name “PHYTOWAX Olive 18 L 57” or waxes obtained by hydrogenating esterified castor oil with cetyl alcohol sold under the name “PHYTOWAX ricin 16L64 and 22L73”, by SOPHIM. Such waxes are described in the application FR-A-2792190.

A silicone wax, in particular sticky, such as a C20-C40 alkyl (hydroxystearyloxy)stearate (the alkyl group comprising 20 to 40 carbon atoms), alone or in a mixture, may be used, in particular a C20-C40 12-alkyl-(12′-hydroxystearyloxy)stearate having the formula (I):

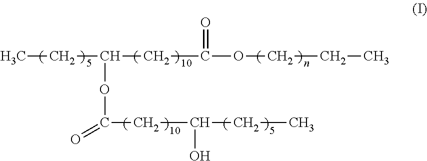

wherein n is an integer ranging from 18 to 38, or a mixture of compounds having the formula (I). Such a sticky wax is particularly sold under the names “KESTER WAX K 82 P” and “KESTER WAX K 80 P” by KOSTER KEUNEN.

Mention can finally be made of microcrystalline waxes, paraffins and ozokerite; polyethylene waxes, waxes obtained by means of Fisher-Tropsch synthesis and waxy copolymers and the esters thereof; silicone waxes and fluorinated waxes.

The coating composition may also comprise at least a pasty compound at 23° C., hydrocarbon or silicon. The pasty fatty substance or substances may be present in the coating composition in a content ranging from 0.1% to 10% by weight, preferably from 0.5% to 5% by weight relative to the total weight of the composition.

For the purposes of the invention, the term “pasty fatty substance” refers to a lipophilic fatty compound having a reversible solid/liquid change of state, having an anisotropic crystalline organization in the solid state, and comprising a liquid fraction and a solid fraction at a temperature of 23° C.

In other words, the initial melting point of the pasty compound may be less than 23° C. The liquid fraction of the pasty compound measured at 23° C. may represent 9 to 97% by weight of the compound. This liquid fraction at 23° C. preferably represents between 15 and 85%, more preferably between 40 and 85% by weight.

The melting point of a solid fatty substance can be measured using a differential scanning calorimeter (DSC), for example the calorimeter sold under the trade name “DSC Q100” by TA Instruments with the “TA Universal Analysis” software, according to the protocol defined hereinabove.

The liquid fraction by weight of the pasty compound at 23° C. is more particularly equal to the ratio of the enthalpy of fusion consumed at 23° C. to the enthalpy of fusion of the pasty compound.

The enthalpy of fusion of the pasty compound is the enthalpy consumed by the compound to change from the solid state to the liquid state. The pasty compound is said to be in the solid state when the entire mass thereof is in solid crystalline form. The pasty compound is said to be in the liquid state when the entire mass thereof is in liquid form.

The enthalpy of fusion of the pasty compound is in particular equal to the area under the curve of the thermogram obtained using a differential scanning calorimeter. The enthalpy of fusion of the pasty compound is the quantity of energy required to change the compound from the solid state to the liquid state. It is expressed in J/g.

The enthalpy of fusion consumed at 23° C. is the quantity of energy required by the sample to change from the solid state to the state presented at 23° C. consisting of a liquid fraction and a solid fraction.

The pasty compound(s) can be in particular chosen from synthetic pasty compounds and fatty substances of plant origin. The pasty compound(s) can be hydrocarbon or silicone.

The pasty compound(s) can be in particular chosen from:
lanolin and its derivatives, such as lanolin alcohol, oxyethylenated lanolins, acetylated lanolin, lanolin esters such as isopropyl lanolate, oxypropylenated lanolins;
vaseline (also called petrolatum),
polyol ethers chosen from C2-C4 pentaerythritol and polyalkylene glycol ethers, fatty alcohol and sugar ethers, and mixtures thereof. For example, mention can be made of pentaerythritol and polyethylene glycol ether comprising 5 oxyethylene patterns (5 OE) (CTFA name: PEG-5 Pentaerythrityl Ether), pentaerythritol and polypropylene glycol ether comprising 5 oxypropylene units (5 OP) (CTFA name: PPG-5 Pentaerythrityl Ether), and the mixtures thereof and more specifically the mixture of PEG-5 Pentaerythrityl Ether, PPG-5 Pentaerythrityl Ether and soybean oil, sold under the name “Lanolide” by VEVY, wherein the ratio of the constituents by weight is 46:46:8: 46% PEG-S Pentaerythrityl Ether, 46% PPG-5 Pentaerythrityl Ether and 8% soybean oil,
polyethers derived from polyetherification between one or a plurality of C2-C100, preferably C2-C50, diols. Of the liposoluble polyethers, ethylene-oxide and/or propylene-oxide copolymers with C6-C30 long-chain alkylene-oxides are particularly considered, more preferably such that the weight ratio of ethylene-oxide and/or propylene-oxide with alkylene-oxides in the copolymer is 5:95 to 70:30. In this family, particular mention may be made of copolymers such as long-chain alkylene-oxides arranged in blocks having a mean molecular weight of 1000 to 10000, for example a polyoxyethylene/polydodecyl glycol block copolymer such as the dodecanediol (22 mol) and polyethylene glycol (45 EO) ethers marketed under the brand ELFACOS ST9 by AKZO NOBEL,
oligomer glycerol esters, especially the esters of diglycerol, with monocarboxylic acids, possibly hydroxylated, linear or branched, saturated or not, preferably saturated, C6-C20, and/or dicarboxylic acids, linear or branched, saturated or not, preferably saturated, C6-C10, in particular condensates of adipic acid and diglycerol, for which a portion of the hydroxyl groups of the glycerols have reacted with a mixture of fatty acids, such as stearic acid, capric acid, stearic acid, isostearic acid and 12-hydroxystearic acid, such as for example bis-diglyceryl polyacyladipate-2 sold under the reference SOFTISAN® 649 by Sasol,
butters of plant origin, such as mango butter, such as the one sold under the reference Lipex 203 by AARHUSKARLSHAMN, shea butter, in particular the one of which the INCI name is Butyrospermum Parkii Butter, such as the one sold under the reference Sheasoft® by AARHUSKARLSHAMN, cupuacu butter (Rain Forest RF3410 from Beraca Sabara), murumuru butter (RAIN FOREST RF3710 from Beraca Sabara), cocoa butter, babassu butter such as the one sold under the name Cropure Babassu SS-(LK) by Croda, as well as orange wax such as, for example, the one sold under the reference Orange Peel Wax by Koster Keunen,
totally or partially hydrogenated plant oils, such as for example hydrogenated soybean oil, hydrogenated coconut oil, hydrogenated rapeseed oil, mixtures of hydrogenated plant oils such as the mixture of hydrogenated plant oils of soybean, coconut, palm and rapeseed, for example the mixture sold under the reference Akogel® by AARHUSKARLSHAMN (INCI name Hydrogenated Vegetable Oil), trans isomerized partially hydrogenated jojoba oil manufactured or sold by Desert Whale under the commercial reference Iso-Jojoba-50®, partially hydrogenated olive oil such as, for example, the compound sold under the reference Beurrolive by Soliance,
hydrogenated castor oil esters, such as dimer dilinoleate hydrogenated castor oil for example RISOCAST-DA-L sold by KOKYU ALCOHOL KOGYO, hydrogenated castor oil isostearate for example SALACOS HCIS (V-L) sold by NISSHIN OIL,
and mixtures thereof.

### Dye

The coating composition can also comprise at least one dye such as powder materials, liposoluble dyes, water-soluble dyes.

The powder dyes can be chosen from pigments and nacres.

The pigments may be white or colored, inorganic and/or organic, coated or uncoated. Of the inorganic pigments, mention may be made of titanium dioxide, optionally surface-treated, zirconium, zinc or cerium oxides, along with iron or chromium oxides, manganese violet, ultramarine blue, chromium hydrate and iron blue. Of the organic pigments, mention may be made of carbon black, D & C type pigments, and lacquers based on cochineal carmine, barium, strontium, calcium, aluminum.

The nacres may be chosen from white pearlescent pigments such as mica coated with titanium, or bismuth oxychloride, colored pearlescent pigments such as titanium mica with iron oxides, titanium mica with iron blue and chromium oxide in particular, titanium mica with an organic pigments of the aforementioned type and pearlescent pigments based on bismuth oxychloride.

The liposoluble colorants are for example Sudan Red, D&C Red 17, D&C Green 6, β-carotene, Sudan Brown, D&C Yellow 11, D&C Violet 2, D&C orange 5, Quinoline Yellow, rocou.

These dyes may be present at a content ranging from 0.01% to 30% by weight, with respect to the total weight of the composition.

### Fibers

The coating compositions can also comprise at least one fiber that allows for an improvement in the lengthening effect.

The term “fiber”, means an object with a length L and diameter D such that L is much greater than D, D being the diameter of the circle in which the section of the fiber is inscribed.

In particular, the ratio L/D (or form factor) is chosen from the range of 3.5 to 2,500, in particular from 5 to 500, and more particularly from 5 to 150.

The fibers that can be used in the coating composition can be fibers of natural or synthetic, mineral or organic origin. They can be short or long, single units or organized for example in braids, hollow or solid. They may be of any shape and in particular of circular or polygonal section (square, hexagonal or octagonal) according to the specific application considered. In particular, their ends are blunt and/or polished in order to prevent injury.

In particular, the fibers have a length ranging from 1 μm to 10 mm, in particular from 0.1 mm to 5 mm and more particularly from 0.3 mm to 3.5 mm. Their section can be comprised in a circle with a diameter ranging from 2 nm to 500 μm, in particular ranging from 100 nm to 100 μm and more particularly from 1 μm to 50 μm. The weight or content of fibers is often given in denier or decitex and represents the weight in grams for 9 km of thread. The fibers according to the invention in particular have a content chosen in the range from 0.15 to 30 deniers and in particular from 0.18 to 18 deniers.

The fibers that can be used in the coating composition can be chosen from rigid or non-rigid fibers, they can be of natural or synthetic, mineral or organic origin.

Moreover, the fibers can be surface-treated or not, coated or uncoated, dyed or not dyed. In terms of fibers that can be used in the coating composition, mention can be made of non-rigid fibers such as polyamide fibers (Nylon®), cellulose fibers or “Rayonne” (RAYON FLOCK RCISE N0003 M04® marketed by the company CLAREMONT FLOCK CORPORATION) or rigid fibers such as the polyimide-amide fibers such as those sold under the names KERMEL®, KERMEL TECH® by the company RHODIA or poly-(p-phenylene-terephtalamide) (or aramide) in particular sold under the name Kevlar® by the company DUPONT DE NEMOURS.

The fibers may be present in the coating composition at a content ranging from 0.01% to 10% by weight, with respect to the weight of the composition, preferably from 0.1% to 5% by weight.

Method of Application

A process for applying a makeup removal composition on keratin fibers of a user, according to the invention, using the requisite for application comprising the device 10 shall now be described.

The keratin fibers are preferably initially coated with a coating composition.

The device 10 is provided with the applicator 14 in idle position, mounted and immobilized on the receptacle 12, the receptacle 12 containing the makeup removal composition.

The first application member 22A is first of all impregnated with the makeup removal composition.

In order to release the applicator 14 from the receptacle 12, the user blocks the rod carrier 18 in rotation with respect to the gripping member 16 by passing the blocking system 24 from its inactive configuration to its active configuration.

For this, the user exerts a radial pressure towards the axis A on each flexible tab 114. Each locking cog 118 is as such inserted into a spline 112, blocking in rotation about the axis A the rod carrier 18 with respect to the gripping member 16.

Simultaneously, the user releases the head for mounting 84 from the neck 30 here by unscrewing the unit formed by the gripping member 16 and the rod carrier 18, and by maintaining the blocking system 24 active. Then, the user extracts the rod carrier 18 and the first application member 22A outside of the receptacle 12 through the opening 28. The squeezing device 32 removes from the first application member 22A, the excess product that accompanies the exit thereof from the receptacle 12.

The user brings the first application member 22A of the makeup removal composition in contact with the keratin fibers preferably already coated with said coating composition.

The user displaces the first application member 22A on the keratin fibers. During this displacement, the mechanical stress of the keratin fibers on the first application member 22A drives in rotation the first application member 22A, the rod 20, and the rod carrier 18 with respect to the gripping member 16.

The displacement is accompanied by the depositing of the makeup removal composition on the keratin fibers. The very low contact between the rod carrier 18 and the application member 16 on the regions 86, 90, decreases the friction between the rod carrier 18 and the gripping member 16.

The friction with the keratin fibers, which are applied on the first application member 22A during the application of the makeup removal composition, very easily cause the rod 20 to rotate with respect to the gripping member 16.

Thus the first application member 22A rolls on the keratin fibers. The force exerted by the user to roll the first application member 22A on the keratin fibers is minimized thanks to the pivot connection with reduced statically indeterminate created by the linear contact regions 86, 90.

As an alternative of the first application member 22A of the device 10, a second application member 22B is shown in FIG. 10.

This second application member 22B differs from the first application member 22A in that the pins of the second row 128 are not arranged staggered with the adjacent pins of the first row 126 and the adjacent pins of the third row 130.

Each row of pins has at least one pin 122 axially positioned, along the longitudinal axis A, at the same position as a pin 122 of each one of the other rows of pins.

In the example of FIG. 10, each pin 122 of the second row 128 is axially positioned, along the longitudinal axis A, at the same position as a pin of the first row 126 and of the third row 130. More generally, each pin 122 of at least one row of pins is axially positioned, along the longitudinal axis A, at the same position as a pin of each one of the other rows of pins.

Each pin 122 of a row that has one pin less that its adjacent rows, is axially positioned, along the longitudinal axis A, at the same position as a pin of each one of its adjacent rows.

As an alternative of the first application member 22A of the device 10, the device 10 comprises a third application member not shown.

This third application member differs from the first application member 22A in that it comprises a foam, the foam being typically able to be impregnated with the makeup removal composition.

The third application member is for example then devoid of pins 122.

According to another embodiment, as an alternative of the first application member 22A of the device 10, the device 10 comprises a fourth application member not shown.

This fourth application member differs from the first application member 22A in that it comprises a threaded rod, the thread being typically able to be impregnated with the coating composition.

Alternatively, the user can selectively block the rotation of the rod carrier 18 with respect to the gripping member 16 at any time, in particular during the application of the product.

Alternatively, the blocking device 19 is configured to authorize a relative translation travel along the axis A of the rod carrier 18 with respect to the gripping member 16, for example a travel less than 10 mm, in particular between 4 mm and 6 mm.

A damping elastic element (not shown) is for example arranged between the rod carrier 18 and the gripping member 16. This elastic element is arranged in such a way that the relative translation along the axis A is carried out, at least in one direction of translation of the rod carrier 18 with respect to the gripping member 16, against an elastic force exerted by the elastic element.

Alternatively, and according to an advantageous arrangement, the blocking system 24 comprises a deformable seal added in the cut around each flexible tab 114, in order to seal the cut at the periphery of each flexible tag 114. Such a seal makes it possible to prevent the insertion of dust into the internal volume 46 of the cover 34.

Alternatively, and according to an advantageous arrangement, the applicator 14 is able to be removably mounted onto the receptacle 12 by means of a snap-fitting or magnetism system, or by any other system that does not require a relative rotation of the applicator 14 with respect to the receptacle 12 in order to separate these two elements.

During the process of application of the makeup removal composition, the user releases the applicator 14 from the receptacle 12, by pulling on the applicator 14 which is separated from the receptacle 12 via simple translation along the axis A.

The examples hereinafter are given by way of illustration and are not intended to limit the invention. Unless mentioned otherwise, the quantities indicated are expressed as a percentage by mass.

### EXAMPLES

### Example 1: Measuring the Makeup Removal Effect of a Makeup Removal Composition with an Applicator 14 Provided with a Second Application Member 22B that is Rotary with Respect to the Gripping Member 16 (Invention)

The support used in each one of the examples (i.e. according to the invention and comparative) is a bar of experimental eyelashes from SPEquation. It is comprised of 60 Caucasian hairs with a height from 5-6 to 13 mm long, arranged over a length of 2 cm. Makeup is applied to each bar with the Volume Effet Faux-Cils mascara twice 15 brush strokes.

Then the inventors remove the makeup using the following three applicator systems:
applicator 14 provided with a second application member 22B (here a Volume Million Cils brush) that is rotary with respect to the gripping member 16 (invention);
applicator provided with an application member similar to the second application member 22B blocked in rotation with respect to a gripping member (comparative 1); and
applicator provided with an application member comprising a fourth application member (i.e. threaded rod) blocked in rotation with respect to a gripping member (comparative 2).

The makeup removal composition is:

| | TRADE | Quantity |
|---|---|---|
| INCI NAME | NAME | (% w/w) |
| Water | | Qs 100 |
| HYDROXYETHYLCELLULOSE | NATROSOL | 2 |
| | 250 MR CS | |
| | (ASHLAND) | |
| HEXYLENE GLYCOL | | 1 |
| DISODIUM COCOAMPHODIACETATE | MIRANOL | 1 |
| | C2M CONC NP | |
| | (SOLVAY) | |
| SODIUM LAURETH-8 SULFATE (and) | RHODAPEX | 0.6 |
| SODIUM LAURETH SULFATE (and) | BSD- 52/FLA2 | |
| MAGNESIUM LAURETH-8 SULFATE | (SOLVAY) | |
| (and) MAGNESIUM LAURETH | | |
| SULFATE (and) SODIUM OLETH | | |
| SULFATE (and) MAGNESIUM OLETH | | |
| SULFATE | | |
| DISODIUM EDTA | | 0.1 |
| PRESERVATIVES | | Qs |

For the example according to the invention, the makeup removal composition is applied in 10 strokes with the applicator 14 provided with the second application member 22B according to the invention.

For the comparative example 1, the makeup removal composition is applied in 10 strokes with the applicator 14 provided with the second application member 22B comparative.

For the comparative example 2, the makeup removal composition is applied in 10 strokes with the applicator provided with the fourth application member (threaded rod) comparative.

The force required to apply the makeup removal in each example is measured.

The measurement of the force is carried out using the Matlab data acquisition and processing software interfaced with an Ametek force measuring system (Sampling frequency f=70 Hz Acquisition time t=30 s).

With regards to the mechanical mounting, the dynamometer is embedded into the fixed frame. The bar of experimental eyelashes is mounted in such a way that the plane of the eyelashes is normal to the direction of the measurement of the force.

The results are as follows:

For the comparative example 1, the average of the maximum values per passage on the 10 strokes is 181 mN;

For the comparative example 2, the average of the maximum values per passage on the 10 strokes is 120.9 mN; and

For the example according to the invention, the average of the maximum values per passage on the 10 strokes is 67 mN.

Thus, it appears that the force to be applied in order to apply the makeup remover is lower with a second application member 22B according to the invention than with a fixed applicator (comparative 1) or with a fourth fixed application member (threaded rod) (comparative 2).

Removing makeup is therefore more comfortable with the applicator 14 provided with the second application member 22B according to the invention.

1. A cosmetic application requisite comprising:
an application member able to be driven in rotation by the keratin fibers with respect to a gripping member; and
makeup removal composition for keratin fibers comprising an aqueous phase and at least one surfactant.

2. The requisite according to claim 1, wherein the driving in rotation of the application member with respect to the gripping member is carried out around a longitudinal axis (A).

3. The requisite according to claim 2, wherein the application member comprises at least one first row of pins and at least one second row (128) of pins, at least one picot of the second row being arranged axially along the longitudinal axis between two adjacent pins of the first row.

4. The requisite according to claim 3, wherein the application member comprises a third row of pins, each pin of the first row being axially positioned, along the longitudinal axis (A), at the same position as a pin of the third row.

5. The requisite according to claim 4, wherein, in projection in a plane perpendicular to the longitudinal axis (A), each pin of the second row of pins is arranged between pins of the first row and pins on the third row, preferably in the middle of the pins of the first row and pins of the third row.

6. The requisite according to claim 2, wherein the application member comprises a plurality of rows of pins, each row having at least one axially positioned pin, along the longitudinal axis (A), at the same position as a pin of each one of the other rows of pins.

7. The requisite according to claim 6, wherein each pin of at least one row of pins is axially positioned, along the longitudinal axis (A), at the same position as a pin of each one of the other rows of pins.

8. The requisite according to claim 3, wherein the rows of pins extend parallel to the longitudinal axis (A).

9. The requisite according to claim 1, wherein the application member comprises a foam.

10. The requisite according to claim 1, wherein the application member is made of elastomer.

11. The requisite according to claim, wherein the makeup removal composition comprises at least 30% by weight of water with respect to the total weight of the composition.

12. The requisite according to claim 1, wherein the surfactant is chosen from:
a) anionic surfactants;
b) amphoteric surfactants;
c) non-ionic surfactants; and
d) mixtures thereof.

13. The requisite according to claim 1, comprising at least one coating composition of keratin fibers.

14. The requisite according to claim 1, comprising an applicator comprising the gripping member, extending along the longitudinal axis, a rod carrier, arranged in the gripping member and mounted freely mobile in rotation about the longitudinal axis (A) with respect to the gripping member, a rod secured to the rod carrier and carrying the application member, wherein the rod carrier as such has a first linear contact region with the gripping member, said first linear contact region being a circumferential linear contact region about the longitudinal axis (A), and a second linear contact region with the gripping member, said second linear contact region being a circumferential linear contact region.

15. The requisite according to claim 14, wherein the first linear contact region and the second linear contact region are separated by a minimum distance l, which is greater than or equal to two times the average diameter between the diameter (D1) of the rod carrier in the first linear contact region and the diameter (D2) of the rod carrier in the second linear contact region.

16. The requisite according to claim 14, wherein each first and second linear contact region has a contact length taken along the longitudinal axis (A) less than 5%, preferably less than 2% of the minimum distance (1) separating along the axis (A) the first linear contact region and the second linear contact region.

17. The requisite according to claim 14, wherein the gripping member comprises an inner sleeve in which the rod carrier is received, the inner sleeve comprising a bulge (76), the first linear contact region extending opposite the bulge.

18. The requisite according to claim 14, wherein the gripping member comprises a peripheral wall about the longitudinal axis (A) and at least one contact tongue radially protruding from the peripheral wall, the or each contact tongue defining a free inner edge directed towards the longitudinal axis (A), the second linear contact region extending opposite the free edge.

19. The requisite according to claim 14, wherein the rod carrier defines a circumferential groove that receives the free edge of the or each contact tongue.

20. The requisite according to claim 14, wherein the gripping member comprises a transverse wall that defines a plurality of radial slots, the radial slots delimiting between them at least two contact tongues located facing the second linear contact region.

21. The requisite according to claim 14, wherein the gripping member comprises a plug and a cover the plug being attached to the cover,
the first linear contact region being delimited opposite the plug,
the second linear contact region being delimited opposite the cover.

22. The requisite according to claim 14, wherein the rod carrier is locked along any rotation following an axis perpendicular to the longitudinal axis (A) of the gripping member.

23. A method for applying a makeup removal composition on keratin fibers, comprising the following steps:
supplying of a cosmetic application requisite according to claim 1;
impregnation of the application member with the makeup removal composition;
bringing the application member in contact with keratin fibers;
driving in rotation of the application member with respect to the gripping member by displacement of the application member on the keratin fibers; and,
depositing of the makeup removal composition on the keratin fibers.

24. The method according to claim 23, wherein the keratin fibers are coated with a coating composition, at least before the bringing the application member in contact with keratin fibers.
